(19) United States
(12) Patent Application Publication
(10) Pub. No.: US 2025/0312272 A1
(43) Pub. Date: Oct. 9, 2025

(54) PHARMACEUTICAL FORMULATIONS FOR ORAL ADMINISTRATION AND METHODS OF MAKING AND USING THE SAME

(71) Applicants: Aaryen Pharma LLC, Granite Falls, NC (US); Brunswick Pharma LLC, North Bergen, NJ (US)

(72) Inventors: Ranga R. Namburi, Granite Falls, NC (US); Venkat Kakani, North Bergen, NJ (US)

(21) Appl. No.: 18/630,363

(22) Filed: Apr. 9, 2024

(51) Int. Cl. A61K 9/00; A61K 31/09; A61K 31/138; A61K 31/167; A61K 31/192; A61K 31/4402; A61K 31/485; A61K 47/36; A61K 47/46

(52) U.S. Cl. CPC A61K 9/0053; A61K 31/09; A61K 31/138; A61K 31/167; A61K 31/192; A61K 31/4402; A61K 31/485; A61K 47/36; A61K 47/46

(57) ABSTRACT

Provided herein are pharmaceutical formulations intended for oral administration comprising one or more active pharmaceutical ingredients, a gum, a syrup, and water. Also provided herein is a pharmaceutical vehicle formulation comprising a gum, a syrup, and a water. Further provided herein are methods of making and methods of using said formulations.

### FIELD OF THE INVENTION

This invention relates to pharmaceutical formulations intended for oral administration comprising one or more active pharmaceutical ingredient (API), a gum, a syrup, and water. The formulation may also comprise a pH buffer and a flavoring agent. This invention further relates to methods of making and methods of using pharmaceutical formulations described herein.

### BACKGROUND OF THE INVENTION

Pharmaceutical suspensions are widely used for oral administration to deliver therapeutic agents, particularly for pediatric and geriatric populations. However, existing suspension formulations comprising only natural ingredients often suffer from issues such as poor stability, poor pourability, or poor patient compliance. Often, the APIs of these suspensions will separate into aggregates that sediment or cream, resulting in poor taste and/or texture. Aggregated APIs in formulations require excessive redispersion (e.g., shaking) prior to consumption, not just to improve palatability but also to ensure that the patient intakes a uniform dose each time.

In addition, some APIs are poorly soluble or sparingly soluble in the pharmaceutical vehicle, so it is useful or necessary to formulate them as a suspension. While heating the vehicle with the poorly soluble or sparingly soluble ingredients may improve their solubility, it is not preferred. Such heating may damage sensitive APIs, thus reducing efficacy, and they may crystallize or aggregate out, by way of sedimentation or creaming, when the formulation is cooled back down to store at room temperature. When the APIs in such formulations crystallize out at room temperature, there is no controlling the resulting particle size. In these formulations, a dispersant is often required in order to keep APIs in suspension in the vehicle.

Current commercially available acetaminophen formulation, where the particles are initially dissolved via heating at the time of manufacturing, form aggregates during storage that cream to the surface of the suspension and are very difficult to redisperse. Many of these formulations are made with agave syrup, which itself is comprised of about 25% of water, and a diluent, additional water, and they cannot suspend the poorly soluble or sparingly soluble acetaminophen API without a suspending or dispersing agent. Further, the pH of these products when measured in laboratories do not meet United States Pharmacopeia (USP) Acetaminophen Oral Suspension monograph specifications. The same USP monograph also requires control of the formation of the 4-aminophenol impurity. Patents describing pharmaceutical syrup formulations include U.S. Pat. Nos. 11,617,795 and 11,931,413, and United States Patent Application Publication No. 2023/0165963.

Poor medication adherence in children is common, as they can be extremely picky on the form or palatability of the medicine. There is no ‘one-size-fits-all’ approach. When selecting medicines for children, it is important to consider the child's age, swallowing ability, ease of administration and accessibility of the product. A suspension with a better mouth feel and taste are obviously preferred. Moreover, medications that primarily comprise natural ingredients as excipients are beneficial as these are more likely to have minimal or no harmful side effects.

Thus, there remains a desire for an improved pharmaceutical suspension formulation that addresses these challenges and enhances therapeutic outcomes.

### SUMMARY OF THE INVENTION

The present invention is based on the finding that pharmaceutical formulations comprising a gum, syrup, and water are suitable for forming suspensions with APIs which are soluble, sparingly soluble, or insoluble in water. Thus, the present invention improves on existing suspension formulations by offering more stable suspensions (e.g., less or no aggregation of the APIs) and better therapeutic outcomes through improved patient satisfaction (e.g., from better palatability and mouthfeel with less requirements for API redispersion). No synthetic preservatives are used in the current invention, which meets microbial attributes as per United States Pharmacopeia for oral suspension or liquid products. The invention further provides formulations comprising all natural components.

Thus, one aspect of the invention relates to a pharmaceutical formulation for oral administration comprising: a) one or more active pharmaceutical ingredient (API) present in a concentration from about 0.02 to about 5 weight/volume percentage concentration (% w/v) of the formulation; b) a gum selected from gum acacia, gum tragacanth, gum almond, gum olibanum, gum ghatti, gum Albizia, and/or Moringa oleifera gum, present in a combined concentration from about 0.2 to about 2.0% w/v of the formulation; c) a syrup selected from agave syrup, tapioca syrup, and/or maple syrup, present in a combined concentration from about 110 to about 130% w/v of the formulation; and d) water present in a concentration from about 5 to about 45% w/v of the formulation. In some embodiments, the gum is gum acacia and the syrup is agave syrup. In some embodiments, the gum is gum acacia and the syrup is agave syrup and tapioca syrup.

Another aspect of the invention relates to a pharmaceutical vehicle for oral administration, comprising: a) a gum selected from gum acacia, gum tragacanth, gum almond, gum olibanum, gum ghatti, gum Albizia, and/or Moringa oleifera gum present in a combined concentration from about 0.2 to about 2.0% w/v of the formulation; b) a syrup selected from agave syrup, tapioca syrup, and/or maple syrup present in a combined concentration from about 110 to about 130% w/v of the formulation; and c) water present in a concentration from about 5 to about 45% w/v of the formulation; wherein the pharmaceutical vehicle is suitable for suspending one or more water soluble, sparingly soluble, and/or water insoluble API. In some embodiments, the gum is gum acacia and the syrup is agave syrup. In some embodiments, the gum is gum acacia and the syrup is agave syrup and tapioca syrup.

Another aspect of the invention relates to a method of making a pharmaceutical formulation and/or a pharmaceutical vehicle as described herein. Thus, one aspect of the invention is a method of making a pharmaceutical vehicle, the method comprising: i) dissolving the gum in the water to form a viscous liquid, wherein the water is heated; and ii) stirring the viscous liquid into the syrup to form a pharmaceutical formulation for oral administration.

Another aspect of the invention is a method for manufacturing a pharmaceutical formulation for oral administration, the method comprising: i) dissolving a gum selected from gum acacia, gum tragacanth, gum almond, gum olibanum, gum ghatti, gum Albizia, and/or Moringa oleifera gum in heated water to form a viscous liquid; ii) stirring one or more micronized API into the viscous liquid to form a uniform dispersion; and iii) stirring the uniform dispersion into a syrup selected from agave syrup, tapioca syrup, and/or maple syrup to form a pharmaceutical formulation for oral administration.

Another aspect of the invention is a method for manufacturing a pharmaceutical formulation for oral administration, the method comprising: i) dissolving a gum selected from gum acacia, gum tragacanth, gum almond, gum olibanum, gum ghatti, gum Albizia, and/or Moringa oleifera gum in a first portion of heated water to form a viscous liquid; ii) stirring a first micronized API into the viscous liquid to form a uniform dispersion; iii) dissolving a second API in a second portion of heated water to form a solution; and iv) stirring the uniform dispersion and the solution into a syrup selected from agave syrup, tapioca syrup, and/or maple syrup to form a pharmaceutical formulation for oral administration.

Another aspect of the invention is a method for manufacturing a pharmaceutical formulation for oral administration, the method comprising: i) dissolving a gum selected from gum acacia, gum tragacanth, gum almond, gum olibanum, gum ghatti, gum Albizia, and/or Moringa oleifera gum in a first portion of heated water to form a viscous liquid; ii) dissolving one or more API in a second portion of heated water to form a solution; and iii) stirring the viscous liquid and the solution into a syrup selected from agave syrup, tapioca syrup, and/or maple syrup to form a pharmaceutical formulation for oral administration.

Another aspect of the invention is a method for manufacturing a pharmaceutical formulation for oral administration, the method comprising: i) dissolving a gum selected from gum acacia, gum tragacanth, gum almond, gum olibanum, gum ghatti, gum Albizia, and/or Moringa oleifera gum in a first portion of heated water to form a viscous liquid; ii) dissolving a first API in a second portion of heated water to form a first solution; iii) dissolving a second API in a third portion of heated water to form a second solution; and iv) stirring the uniform dispersion, the first solution, and the second solution into a syrup selected from agave syrup, tapioca syrup, and/or maple syrup to form a pharmaceutical formulation for oral administration.

Another aspect of the invention relates to methods of administering/delivering a pharmaceutical formulation as described herein. Thus, one aspect of the invention is a method of delivering an API to a subject in need thereof, the method comprising orally administering to the subject an effective amount of a pharmaceutical formulation described herein, thereby delivering the API to the subject.

Another aspect of the invention is a method of treating pain in a subject in need thereof, the method comprising orally administering to the subject a therapeutically effective amount of a pharmaceutical formulation described herein, thereby treating pain in the subject.

Another aspect of the invention is a method of treating a respiratory illness in a subject in need thereof, the method comprising orally administering to the subject a therapeutically effective amount of a pharmaceutical formulation described herein, thereby treating the respiratory illness in the subject.

Another aspect of the invention is a method of reducing fever in a subject in need thereof, the method comprising orally administering to the subject a therapeutically effective amount of a pharmaceutical formulation described herein, thereby reducing fever in the subject.

These and other aspects of the invention are set forth in more detail in the description of the invention below.

### BRIEF DESCRIPTION OF THE DRAWINGS

FIG. 1 is an image showing the comparison of pharmaceutical formulations for oral administration comprising acetaminophen as the API, a commercially available pharmaceutical formulation is seen in the left bottle, and a pharmaceutical formulation as described herein is seen in the right bottle.

FIG. 2, panel A is an image showing the comparison pharmaceutical formulations for oral administration comprising dextromethorphan hydrobromide and guaifenesin as the APIs, a commercially available pharmaceutical formulation is seen in the left vial, and a pharmaceutical formulation as described herein is seen in the right vial. FIG. 2, panel B is an image showing the comparison pharmaceutical formulations for oral administration comprising dextromethorphan hydrobromide and guaifenesin as the APIs after three months of storage at room temperature, a commercially available pharmaceutical formulation is seen in the left vial, and a pharmaceutical formulation as described herein is seen in the right vial.

### DETAILED DESCRIPTION OF THE INVENTION

The present invention is explained in greater detail below. This description is not intended to be a detailed catalog of all the different ways in which the invention may be implemented, or all the features that may be added to the instant invention. For example, features illustrated with respect to one embodiment may be incorporated into other embodiments, and features illustrated with respect to a particular embodiment may be deleted from that embodiment. In addition, numerous variations and additions to the various embodiments suggested herein will be apparent to those skilled in the art in light of the instant disclosure which do not depart from the instant invention. Hence, the following specification is intended to illustrate some particular embodiments of the invention, and not to exhaustively specify all permutations, combinations and variations thereof.

Unless the context indicates otherwise, it is specifically intended that the various features of the invention described herein can be used in any combination. Moreover, the present invention also contemplates that in some embodiments of the invention, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention. All publications, patent applications, patents, and other references mentioned herein are incorporated by reference in their entirety. In case of a conflict in terminology, the present specification is controlling.

All publications, patent applications, patents, and other references mentioned herein are incorporated by reference in their entirety.

As used in the description of the invention and the appended claims, the singular forms “a,” “an,” and “the” are intended to include the plural forms as well, unless the context clearly indicates otherwise.

Also as used herein, “and/or” refers to and encompasses any and all possible combinations of one or more of the associated listed items, as well as the lack of combinations when interpreted in the alternative (“or”).

The term “about,” as used herein when referring to a measurable value such as an amount of polypeptide, dose, time, temperature, enzymatic activity or other biological activity and the like, is meant to encompass variations of ±10%, ±5%, ±1%, ±0.5%, or even ±0.1% of the specified amount.

As used herein, the transitional phrase “consisting essentially of” (and grammatical variants) is to be interpreted as encompassing the recited materials or steps and those that do not materially affect the basic and novel characteristic(s) of the claimed invention. Thus, the term “consisting essentially of” as used herein should not be interpreted as equivalent to “comprising.”

The term “enhance” or “increase” refers to an increase in the specified parameter of at least about 1.25-fold, 1.5-fold, 2-fold, 3-fold, 4-fold, 5-fold, 6-fold, 8-fold, 10-fold, twelve-fold, or even fifteen-fold and/or can be expressed in the enhancement and/or increase of a specified level and/or activity of at least about 1%, 5%, 10%, 15%, 25%, 35%, 40%, 50%, 60%, 75%, 80%, 90%, 95% or more.

The term “inhibit” or “reduce” or grammatical variations thereof as used herein refers to a decrease or diminishment in the specified level or activity of at least about 15%, 25%, 35%, 40%, 50%, 60%, 75%, 80%, 90%, 95% or more. In particular embodiments, the inhibition or reduction results in little or essentially no detectible activity (at most, an insignificant amount, e.g., less than about 10% or even 5%).

A “therapeutically effective” amount as used herein is an amount that provides some improvement or benefit to the subject. Alternatively stated, a “therapeutically effective” amount is an amount that will provide some alleviation, mitigation, or decrease in at least one clinical symptom in the subject (e.g., in the case of a respiratory illness, reduced coughing, reduced fever, reduced nasal mucus discharge, or increase in survival time). Those skilled in the art will appreciate that the therapeutic effects need not be complete or curative, as long as some benefit is provided to the subject.

A “subject” may be any vertebrate organism in various embodiments. A subject may be individual to whom an agent is administered, e.g., for experimental, diagnostic, and/or therapeutic purposes or from whom a sample is obtained or on whom a procedure is performed. In some embodiments a subject is a mammal. A mammalian subject may include, but is not limited to, a laboratory animal (e.g., a rat, mouse, guinea pig, rabbit, primate, etc.), a farm or commercial animal (e.g., cattle, pig, horse, goat, donkey, sheep, etc.), or a domestic animal (e.g., cat, dog, ferret, gerbil, hamster, etc.). In some embodiments, a mammalian subject may be a primate, or a non-human primate (e.g., a chimpanzee, baboon, macaque (e.g., rhesus macaque, crab-eating macaque, stump-tailed macaque, pig-tailed macaque), monkey (e.g., squirrel monkey, owl monkey, etc.), marmoset, gorilla, etc.). In some embodiments, a mammalian subject may be a human. In some embodiments a human subject is a neonate, child, adult, or geriatric subject. In some embodiments a human subject is 1, 2, 3, 4, 5, 6, 7, 8, 9, 10, 11, or 12 months old. In some embodiments a human subject is 1, 2, 3, 4, 5, 6, 7, 8, 9, 10, 11, 12, 13, 14, 15, 16, 17, 18, 19, or 20 years old. In some embodiments a human subject is at least 60, 70, 80, or 90 years old.

A “subject in need” of the methods of the invention can be any subject known or suspected of having a disease or a disorder that is treatable by pharmaceutical formulation of the invention.

In some embodiments, the subject in need requires or would benefit from a pharmaceutical formulation designed for oral administration instead of any other route of administration for any reason known to those in the art (e.g., increased compliance to medication regimens, increased ease of administration, reduced complication rate compared to other administration methods, etc., e.g., subjects unable to swallow tablets or capsules, such as children, the elderly, and subjects with swallowing issues). In some embodiments, the disease or disorder is a respiratory illness, such as a viral respiratory infection (e.g., an influenza infection, a coronavirus (e.g., COVID-19) infection, a respiratory syncytial virus (RSV) infection, a rhinovirus infection, an adenovirus infection, and/or a parvovirus infection). In some embodiments, the disease or disorder is a fever. In some embodiments, the disease or disorder is inflammation (e.g., localized inflammation or systemic inflammation). In some embodiments, the disease or disorder is pain (e.g., acute pain or chronic pain).

“Treat,” “treating” and similar terms as used herein in the context of treating a subject refer to providing medical and/or surgical management of a subject. Treatment may include, but is not limited to, administering an agent or composition (e.g., a pharmaceutical composition) to a subject. Treatment is typically undertaken in an effort to alter the course of a disease (which term is used to indicate any disease, disorder, syndrome, or undesirable condition warranting or potentially warranting therapy) in a manner beneficial to the subject. The effect of treatment may include reversing, alleviating, reducing severity of, curing, inhibiting the progression of, and/or reducing the likelihood of recurrence of the disease or one or more symptoms or manifestations of the disease. A therapeutic agent may be administered to a subject who has a disease or is at increased risk of developing a disease relative to a member of the general population. In some embodiments a therapeutic agent may be administered to a subject who has had a disease but no longer shows evidence of the disease. The agent may be administered e.g., to reduce the likelihood of recurrence of evident disease. A therapeutic agent may be administered prophylactically, i.e., before development of any symptom or manifestation of a disease. “Prophylactic treatment” refers to providing medical and/or surgical management to a subject who has not developed a disease or does not show evidence of a disease in order, e.g., to reduce the likelihood that the disease will occur, delay the onset of the disease, or to reduce the severity of the disease should it occur. The subject may have been identified as being at risk of developing the disease (e.g., at increased risk relative to the general population or as having a risk factor that increases the likelihood of developing the disease.

Grammatical variations of “administer,” “administration,” and “administering” to a subject include any route of introducing or delivering to a subject an agent. Administration can be carried out by any suitable route, including oral administration. In some embodiments, the administration is oral administration. “Concurrent administration,” “administration in combination,” “simultaneous administration,” or “administered simultaneously” as used herein, means that the compounds are administered at the same point in time, overlapping in time, or one following the other. In the latter case, the two compounds are administered at times sufficiently close that the results observed are indistinguishable from those achieved when the compounds are administered at the same point in time. “Systemic administration” refers to the introducing or delivering to a subject an agent via a route which introduces or delivers the agent to extensive areas of the subject's body (e.g., greater than 50% of the body), for example through entrance into the circulatory or lymph systems. By contrast, “local administration” refers to the introducing or delivery to a subject an agent via a route which introduces or delivers the agent to the area or area immediately adjacent to the point of administration and does not introduce the agent systemically in a therapeutically significant amount. For example, locally administered agents are easily detectable in the local vicinity of the point of administration but are undetectable or detectable at negligible amounts in distal parts of the subject's body. Administration includes self-administration and the administration by another.

“Prevent” or “preventing” or “prevention” refer to prevention or delay of the onset of the disease and/or disorder, and/or a decrease in the severity of the disease and/or disorder in a subject relative to the severity that would develop in the absence of the methods of the invention. The prevention can be complete, e.g., the total absence of pain, fever, and/or a respiratory illness. The prevention can also be partial, such that the occurrence or severity of pain, fever, and/or a respiratory illness in a subject is less than that which would have occurred without the present invention.

“Suspension” or “pharmaceutical suspension” as used herein refers to a formulation where one or more solid ingredients (e.g., APIs) is poorly soluble, sparingly soluble, or insoluble in the vehicle and/or the solvent and are thus suspended in the solution with the aid of one or more excipients (e.g., a dispersant or emulsifier). In some embodiments, the type of pharmaceutical suspension is named based on the size of the solid particles: colloidal suspension (<1 micron), coarse suspension (>1 micron), or nanosuspension (about 10 nm). In some embodiments, the suspension comprises one or more poorly soluble, sparingly soluble, or insoluble solid particles (e.g., API particles) having a specific range of particle sizes (e.g., the internal phase) which are uniformly dispersed throughout a vehicle and/or a solvent (e.g., the external phase). In some embodiments, the external phase is aqueous.

The terms “natural ingredient”, “natural product”, and the like are used herein to mean ingredients/products which can be found in nature (e.g., isolated, extracted, and/or derived from a microorganism such as yeast or a bacterium, a plant, or an animal). In some embodiments, the natural ingredient may be a product that is extracted from a plant (e.g., a gum or a syrup from the sap). In some embodiments, the natural ingredient may be further processed and/or purified for use in a pharmaceutical formulation as described herein. In some embodiments, the further processing and/or purification may include, but is not limited to, dehydrating, boiling, reducing, separating, distilling, drying, emulsifying, isolating, grinding, and/or mashing.

### Pharmaceutical Formulations

One aspect of the invention relates to a pharmaceutical formulation for oral administration comprising: a) one or more active pharmaceutical ingredient (API) present in a concentration from about 0.02 to about 5 weight/volume percentage concentration (% w/v) of the formulation; b) a gum present in a concentration from about 0.2 to about 2.0% w/v of the formulation; c) a syrup present in a concentration from about 110 to about 130% w/v of the formulation; and d) water present in a concentration from about 5 to about 45% w/v of the formulation. In some embodiments, the formulation further comprises e) a flavoring agent. In some embodiments, the flavoring agent is present a concentration from about 0.1 to about 1% w/v of the formulation. In some embodiments, the flavoring agent is blueberry flavor, strawberry flavor, raspberry flavor, blackberry flavor, cherry flavor, apple flavor, lemon flavor, lime flavor, or other similar flavors. In some embodiments, the gum prevents separation of the flavoring agent formulation during storage. In some embodiments, the formulation further comprises f) a pH buffer. In some embodiments, the pH buffer is present a concentration from about 0.1 to about 1% w/v of the formulation. In some embodiments, the pH buffer is citrus extract or grape seed extract. In some embodiments, citrus extract or grape seed extract is used to adjust the pH of the formulation. In some embodiments, the pH of the formulation is from about 2 to about 7 (e.g., from about 2 to about 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, to about 7.0 or any range therein), from about 4 to about 6.9, or from about 2.3 to about 6.5. In some embodiments, the viscosity of the formulation is less than about 1400 centipoise (e.g., less than about 1400, 1300, 1200, 1100, 1000, 900, 800, 700, 600, 550, 500, 450, 400, 350, 300, 250, or about 200 centipoise) at about 22° C. (e.g., at about 19, 19.5, 20, 20.5, 21, 21.5, 22, 22.5, 23, 23.5, 24, 24.5, or about 25° C.), less than about 900 centipoise at about 22° C., or less than about 450 centipoise at about 22° C. In some embodiments, the gum, the syrup, the flavoring agent, and/or the pH buffer are natural ingredients. In some embodiments, the total water content of the formulation is from about 30% w/v to about 45% w/v (e.g., the total water content includes the water content of the syrup in addition to the water added to the formulation).

In some embodiments, the one or more API is water insoluble (e.g., practically insoluble or very slightly soluble (see Table 1)), sparingly soluble in water (e.g., slightly soluble and/or sparingly soluble, e.g., from about 100 to 1000 or about 30 to about 100 parts of solvent are required for one part of solute), or is water soluble (e.g., soluble, freely soluble and/or very soluble, e.g., from about 10 to about 30 or about 1 to 10 or less than 1 parts of solvent are required for one part of solute). In some embodiments, each of the one or more API is a micronized API. The API may be micronized by any method known in the art, e.g., by jet milling, ball milling, spray drying, controlled crystallization, and/or the like. In some embodiments, the micronized API has a DV90 particle size from about 25 μm to about 100 μm (e.g., about 25, 30, 35, 40, 45, 50, 55, 60, 65, 70, 75, 80, 85, 90, 95, or about 100 μm or any range therein). In some embodiments, the micronized API has a DV90 particle size from about 1 μm to about 24 μm (e.g., 1, 2, 3, 4, 5, 6, 7, 8, 9, 10, 11, 12, 13, 14, 15, 16, 17, 18, 19, 20, 21, 22, 23, or about 24 μm or any range therein). In some embodiments, the micronized API has a median particle size of about 1, 2, 3, or 4 μm to about 5, 6, 7, 8, 9, 10, 11, 12 13, 14, 15, 16, 17, 18, 19, 20, 21, 22, 23, or 24 μm or any range therein. In some embodiments, the micronized API has a median particle size of about 25, 30, 35, 40, or 45 μm to about 50, 55, 60, 65, 70, 75, 80, 85, 90, 95, or 100 μm or any range therein. In some embodiments, the one or more water insoluble or sparingly soluble in water API is suspended in the formulation. In some embodiments, the one or more suspended API is easily redispersible into the formulation. In some embodiments, the one or more easily redispersible API allows for repeated, accurate dosage from a multiple dosage container. In some embodiments, the one or more API is an analgesic, a non-steroidal anti-inflammatory, an antihistamine, a cough suppressant, an expectorant, and/or a fever reducer. In some embodiments, the one or more API is water insoluble or sparingly soluble in water, e.g., acetaminophen and/or guaifenesin. In some embodiments, the one or more API is water soluble, e.g., dextromethorphan hydrobromide, diphenhydramine hydrochloride, and/or chlorpheniramine maleate. In some embodiments, the API is present in the formulation as a suspension.

TABLE 1
Descriptive terms for solubility.

| Descriptive term | Part of solvent required per part of solute |
|---|---|
| Very soluble | Less than 1 |
| Freely soluble | From 1 to 10 |
| Soluble | From 10 to 30 |
| Sparingly soluble | From 30 to 100 |
| Slightly soluble | From 100 to 1,000 |
| Very slightly soluble | From 1,000 to 10,000 |
| Practically insoluble | 10,000 and over |

In some embodiments, the gum is a complex carbohydrate polymer comprising galactose, arabinose, rhamnose and/or uronic acid. In some embodiments, the gum is a plant-based gum (e.g., a natural gum) derived from the hardened sap of a plant. In some embodiments, the gum is selected from gum acacia (i.e., gum arabic), gum tragacanth, gum almond, gum olibanum, gum ghatti, gum Albizia, and/or Moringa oleifera gum. In some embodiments, the gum is present in a combined concentration from about 0.2 to about 2.0% w/v (e.g., about 0.2, 0.3, 0.4, 0.5, 0.6, 0.7, 0.8, 0.9, 1.0, 1.1, 1.2, 1.3, 1.4, or about 1.5% w/v or 2.0% any range therein) of the formulation. In some embodiments, the gum is gum acacia. In some embodiments, the gum acacia is obtained from (e.g., processed from) Senegalia senegal or Vachellia seyal. In some embodiments, the gum is used as an emulsifying agent, a stabilizing agent, a thickening agent, and/or a microencapsulation agent. In some embodiments, the gum is a dispersant and/or suspending agent for one or more API (e.g., water insoluble or sparingly soluble API) in the formulation. In some embodiments, the gum prevents separation of the flavoring agent formulation during storage.

In some embodiments, the syrup is a sticky and/or tacky liquid comprising sucrose, fructose, and/or glucose. In some embodiments, the syrup is a plant-based syrup (e.g., a natural syrup) obtained from the juice (e.g., sap) of a plant. In some embodiments, the syrup is selected from agave syrup, tapioca syrup, and/or maple syrup. In some embodiments, the syrup is present in a combined concentration from about 110 to about 130% w/v (e.g., about 110, 111, 112, 113, 114, 115, 116, 117, 118, 119, 120, 121, 122, 123, 124, 125, 126, 127, 128, 129, or about 130% w/v or any range therein) of the formulation. In some embodiments, the syrup is agave syrup and tapioca syrup. In some embodiments, the syrup is agave syrup (e.g., derived from the sap of the agave plant). In some embodiments, the agave syrup is obtained from (e.g., processed from) Agave salmiana and Agave tequilana (e.g., Agave tequilana Weber Blue variety). In some embodiments, the syrup is a vehicle in the formulation. In some embodiments, the density of the syrup (e.g., agave syrup) is from about 0.5 to about 2 g/mL (e.g., about 0.5, 0.75, 1, 1.25, 1.5, 1.75, or about 2 g/mL or any range therein). In some embodiments, the density of the syrup is about 1.38 g/mL. In some embodiments, the syrup comprises from about 22-28% weight by weight (w/w) of water (e.g., about 22, 22.5, 23, 23.5, 24, 24.5, 25, 25.5, 26, 26.5, 27, 27.5, or about 28% w/w of water).

Another aspect of the invention relates to a pharmaceutical vehicle for oral administration, comprising: a) a gum selected from gum acacia, gum tragacanth, gum almond, gum olibanum, gum ghatti, gum Albizia, and/or Moringa oleifera gum present in a combined concentration from about 0.2 to about 2.0% w/v of the formulation; b) a syrup selected from agave syrup, tapioca syrup, and/or maple syrup present in a combined concentration from about 110 to about 130% w/v of the formulation; and c) water present in a concentration from about 5 to about 45% w/v of the formulation; wherein the pharmaceutical vehicle is suitable for suspending one or more water soluble, sparingly soluble, and/or water insoluble API. In some embodiments, the pharmaceutical vehicle comprises b) agave syrup present in a concentration of about 127% w/v; c) gum acacia present in a concentration of about 1.0% w/v; and d) water present in a concentration of about 8.0% w/v. A pharmaceutical vehicle of the present invention is suitable for suspending, dissolving, emulsifying, or otherwise being a carrier for any API that may be administered orally to a subject in need thereof.

In some embodiments, a pharmaceutical formulation and/or pharmaceutical vehicle of the invention meets or exceeds guidelines and testing parameters for pharmaceuticals, suspensions, and/or liquid dosage forms as set forth by the USP and/or similar agencies (e.g., the European Pharmacopeia). In some embodiments, the testing includes, but is not limited to, antimicrobial effectiveness testing (e.g., USP<51>), uniformity of dosage units (e.g., USP<905>), suspensions and redispersibility, general pharmaceutical compounding for non-sterile preparations (e.g., USP<795>), and general pharmaceutical compounding for sterile preparations (e.g., USP<797>).

### Methods of Making

One aspect of the invention relates to a method of making (e.g., manufacturing) a pharmaceutical formulation and/or a pharmaceutical vehicle described herein.

In some embodiments, the method of making a pharmaceutical vehicle comprises: i) dissolving the gum in the water to form a viscous liquid, wherein the water is heated; and ii) stirring the viscous liquid into the syrup to form a pharmaceutical vehicle for oral administration. In some embodiments, the water is heated to about 40° C. or to about 60° C. (e.g., about 40, 45, 50, 55, or about 60° C. or any range therein). In some embodiments, the gum is gum acacia and the syrup is agave syrup and/or tapioca syrup.

In some embodiments, the method of manufacturing a pharmaceutical formulation comprises: i) dissolving a gum selected from gum acacia, gum tragacanth, gum almond, gum olibanum, gum ghatti, gum Albizia, and/or Moringa oleifera gum in heated water to form a viscous liquid; ii) stirring one or more micronized sparingly water soluble or insoluble API into the viscous liquid to form a uniform dispersion; and iii) stirring the uniform dispersion into a syrup selected from agave syrup, tapioca syrup, and/or maple syrup to form a pharmaceutical formulation for oral administration.

In some embodiments, the method of manufacturing a pharmaceutical formulation comprises: i) dissolving a gum selected from gum acacia, gum tragacanth, gum almond, gum olibanum, gum ghatti, gum Albizia, and/or Moringa oleifera gum in a first portion of heated water to form a viscous liquid; ii) stirring a first API into the viscous liquid to form a uniform dispersion; iii) dissolving a second API in a second portion of heated water to form a solution; and iv) stirring the uniform dispersion and the solution into a syrup selected from agave syrup, tapioca syrup, and/or maple syrup to form a pharmaceutical formulation for oral administration.

In some embodiments, the method of manufacturing a pharmaceutical formulation comprises: i) dissolving a gum selected from gum acacia, gum tragacanth, gum almond, gum olibanum, gum ghatti, gum Albizia, and/or Moringa oleifera gum in a first portion of heated water to form a viscous liquid; ii) dissolving one or more API in a second portion of heated water to form a solution; and iii) stirring the viscous liquid and the solution into a syrup selected from agave syrup, tapioca syrup, and/or maple syrup to form a pharmaceutical formulation for oral administration. In some embodiments, the method of manufacturing a pharmaceutical formulation comprises: i) dissolving a gum selected from gum acacia, gum tragacanth, gum almond, gum olibanum, gum ghatti, gum Albizia, and/or Moringa oleifera gum in a first portion of heated water to form a viscous liquid; ii) dissolving a first API in a second portion of heated water to form a first solution; iii) dissolving a second API in a third portion of heated water to form a second solution; and iv) stirring the uniform dispersion, the first solution, and the second solution into a syrup selected from agave syrup, tapioca syrup, and/or maple syrup to form a pharmaceutical formulation for oral administration.

In some embodiments, the method of making a pharmaceutical vehicle and/or manufacturing a pharmaceutical formulation further comprises stirring a pH buffer and/or a flavoring agent into the syrup. In some embodiments, the heated water, the first portion of the heated water, the second portion of the heated water, and/or the third portion of the heated water are about 40° C. to about 60° C. (e.g., about 40, 45, 50, 55, or about 60° C. or any range therein).

### Methods of Use

One aspect of the invention relates to a method of delivering an API to a subject in need thereof, the method comprising orally administering to the subject an effective amount of a pharmaceutical formulation as described herein, thereby delivering the API to the subject. Another aspect of the invention relates to a method of treating pain in a subject in need thereof, the method comprising orally administering to the subject a therapeutically effective amount of a pharmaceutical formulation as described herein, thereby treating pain in the subject. Another aspect of the invention relates to a method of treating a respiratory illness in a subject in need thereof, the method comprising orally administering to the subject a therapeutically effective amount of a pharmaceutical formulation as described herein, thereby treating the respiratory illness in the subject. Another aspect of the invention relates to a method of reducing fever in a subject in need thereof, the method comprising orally administering to the subject a therapeutically effective amount of a pharmaceutical formulation as described herein, thereby reducing fever in the subject.

In some embodiments, a pharmaceutical formulation of the present invention is administered to the subject in an amount of about 0.1 mL to about 60 mL (e.g., about 0.1, 0.2, 0.3, 0.4, 0.5, 0.6, 0.7, 0.8, 0.9, 1, 1.5, 2, 2.5, 3, 3.5, 4, 4.5, 5, 6, 7, 8, 9, 10, 15, 20, 25, 30, 35, 40, 45, 50, 55, or about 60 mL or any range therein). In some embodiments, a pharmaceutical formulation of the present invention is administered to the subject in an amount such that the one or more API in the formulation (e.g., in suspension) is administered to the subject in a therapeutically effective amount. The amount of the pharmaceutical formulation administered to a subject will vary from subject to subject, depending on the nature of the disclosed formulations, the API in the formulation (e.g., in suspension), the species, gender, age, weight and general condition of the subject, the mode of administration, and the like. Effective dosages and schedules for administering the compositions may be determined empirically, and making such determinations is within the skill in the art. The dosage ranges for the administration of the disclosed compositions are those large enough to produce the desired effect (e.g., to reduce pain, treat a respiratory illness, reduce fever, etc.). The dosage should not be so large as to outweigh benefits by causing extensive or severe adverse side effects, such as unwanted cross-reactions, anaphylactic reactions, and the like, although some adverse side effects may be expected. The dosage can be adjusted by the individual clinician in the event of any counterindications.

Having described the present invention, the same will be explained in greater detail in the following examples, which are included herein for illustration purposes only, and which are not intended to be limiting to the invention.

### EXAMPLES

### Example 1

### Background

Pharmaceutical suspensions include an external phase comprising the vehicle (e.g., base) and an internal phase comprising the API, which is homogeneously dispersed in the entire external phase. Suspensions have some advantages over other dosage forms due to their liquid character, especially for patients having problems swallowing capsules or tablets, such as children or geriatric patients. Pharmaceutical suspensions react like other disperse systems and generally need to be stabilized because they are often not thermodynamically stable. One of the critical challenges in the pharmaceutical industry is to develop a stable suspension that aids in increasing the shelf life of the drug product. Therefore, a suitable dosage form of suspending agent or stabilizer is necessary to reduce the rate of sedimentation and allow easy redispersion of any particulate matter for uniformity and stability in the suspending medium. It may be preferable to make suspensions with simple dispersion techniques that require no heating of the API that is poorly or sparingly soluble in water, as those that are dissolved by heating in the vehicle may separate back out as crystalline aggregates upon storage at room temperature. API particle size is controlled to keep the particles in suspended form, prior to incorporation into vehicle without the heating process.

Excipients as suspending agents are pharmacologically inactive materials that are used in pharmaceutical formulations as carriers for the active ingredients. Suspending agents possess an important role in the drug industry due to maintaining the safety, efficacy, and stability of APIs. So, the selection of the suitable suspending agent or dispersant in the development of an appropriate pharmaceutical suspension is very important because the physical stability of a suspension medium is more dependent on the physical properties of the suspending agent than the API's physical characteristics. Suspending agents can be broadly categorized as (1) inorganic materials, (2) synthetic compounds, or (3) polysaccharides. Recently, scientists have paid special attention to polysaccharides because of their natural origin (plant, animal, microbe, and algal), reliability, and sustainability. Among the different sources of polysaccharides, plant polysaccharides are widely used as suspending agents in pharmaceutical formulations due to their specific properties such as biodegradability, low cost, abundancy, biocompatibility, water-solubility, and nontoxicity. The main sources of plant polysaccharides are gums, starches, and mucilages with a wide range of pharmaceutical applications. Plant gums and mucilage are bioavailable, nontoxic, and low-cost polysaccharides that also possess the ability to bind water and form gels due to their hydrophilic molecules in their structure, making them great natural alternatives to be utilized in pharmaceutical suspensions as suspending agents rather than synthetic polymers. Gum acacia (e.g., gum arabic) and gum tragacanth are the most frequently medicinally applied plant-derived gums. Starches, due to their weak structure, need to be modified to be a suitable pharmaceutical excipient. Modified starches, mainly chemically modified ones, have been utilized as suspending agents in pharmaceutical suspensions and their promising suspending activity in pharmaceutical suspensions proved their potential to be used as a commercial suspending agent.

Developing a stable suspension is challenging, as suspensions are thermodynamically unstable. Accordingly, it is beneficial to introduce a stabilizer or a suspending agent which reduces aggregation of the solutes, reducing either settling or creaming, and further permits easy redispersion of any settled particulate matter. Such suspending agents can work both by protective colloidal action and by increasing the consistency of the suspending medium. The selection of the proper suspending agents is important in the development of a suitable pharmaceutical suspension since the type and nature of the suspending agents have significant impact on the physical stability of the suspension. Pharmaceutical suspensions are also liable to have ingredients separate out and change color over time. Thus, these formulations often include instructions to shake well before use. However, if these formulations lack a dispersing agent in the suspension, adequate redispersion to have consistent dosages is often very difficult to achieve.

Chemical testing is important to ensure the stability, safety, and quality of pharmaceutical suspensions. Analyzing the appearance of the phases in a suspension is a method used to evaluate a group of parameters in the dispersed phase and dispersion medium, such as: particle size distribution, clarity of syrup, viscosity of gum dispersion, and quality control of the water used for preparation of the dispersion phase.

The stability of a suspension depends strongly on the sedimentation rate of the dispersed phase which is itself dependent on the viscosity of the dispersion medium. Thus, the viscosity should be set in an optimum range in order to ensure a stable and redispersable suspension, and it is generally best measured both before and after mixing with a dispersed phase. If any difference between these values is found, then there should be some corrective action to reach the optimized viscosity. The pH of the suspension phases also has an effect on its stability. Therefore, each phase's pH must be monitored before and after mixing to make sure that the optimum pH range is achieved.

The drug particles in the dispersed phase have a key role in the suspension stability, and micronization of a sparingly water soluble or poorly water soluble drug may be required to achieve a proper suspension. Therefore, it is necessary to analyze the drug particle size range. In case of the presence of any deviation from the optimum particle size required to maintain the suspension, stricter monitoring of the micronization step must be performed.

Pourability tests are performed after mixing the suspension phases so as to check whether the final product can be easily poured from the storage container, lest the suspension cause problems (e.g., measuring dosages, palatability, etc.) for the patient.

Finally, homogeneity of the final product should be checked. For this purpose, samples are withdrawn after different stages of the product preparation to check if the API is uniformly distributed throughout the dosage form. The mixing step plays a vital role in preparing the product with a reliable dosage formulation.

### Ingredients

APIs can be micronized before incorporated into the formulation so as to have a better control of particle size. This improves dispersion and suspension in the vehicle selected.

Gum acacia is known to have unique emulsifying properties and can be beneficial in treating such health problems as obesity, heart disease, and diabetes, as well as various renal and colon related problems. The emulsifying ability is an important property of acacia gum which enables its use as emulsifier in flavors and oils, and is due to presence of proteinaceous material in its molecular structure which stabilizes the emulsion. Flavor constituents may separate out in pharmaceutical vehicles, and the usage of gum acacia can counteract this to keep them in dispersed phase.

Agave syrup, also known as agave nectar, is a sweetener derived from the sap of the agave plant. The grading of agave syrup typically refers to its color and flavor profile, which can vary depending on factors such as processing methods and the specific type of agave used. One such useful type of agave syrup is made from 100% Agave tequilana Weber Blue variety. It has a fructose content above 84%, a low glycemic index of 17.1 (as tested by the Glycemic Index Laboratories) and is highly soluble in water. Light agave syrup grade often has a light color and mild flavor. It undergoes minimal processing, which helps retain its natural sweetness and delicate taste. Light agave syrup is often used as a substitute for sugar in recipes where a neutral flavor is desired. Currently formulated suspensions with agave syrup tend to cream or crystallize out, and it is difficult to redisperse the API. Often a change of color of the formulation was observed over time. Current methods of making formulations with agave syrup require heating the syrup with the API, however this may cause the discoloration or other physical instability.

Color stability of a pharmaceutical suspension is an important quality attribute. Specifically, mixing under heat is often needed to combine a poorly or sparingly water-soluble API, such as acetaminophen or guaifenesin, in agave syrup with small percentage of water, but this may have an impact on the color stability of the final formulation. The color of pharmaceutical products is very sensitive and can be affected by different processing operations, specifically heat processing. It is thus preferable to avoid heating of the main vehicle to solubilize APIs with poor solubility. Without wishing to be bound by any particular theory, gum acacia, due to its ability to maintain color stability in a solution, may be incorporated into a suspension that tends to change color upon storage.

pH buffers and known flavoring ingredients can be included in the formulations to further improve stability and/or palatability. For example, natural citrus extract containing polyphenols and ascorbic acid, among other ingredients, may improve the pH environment enough to stabilize the product during storage.

Water can be used as a diluent for the gum (e.g., gum acacia), and/or for solubilizing any water-soluble APIs. However, the water content should be controlled below 50% v/v, as too much water could reduce the efficacy, stability, or palatability (e.g., through having to administer too much volume) of the pharmaceutical formulation. Other sources of water in the formulations may be from the syrup (e.g., agave syrup). For example, agave syrup water content is between 24-26% weight by weight, and should be considered when making the pharmaceutical formulations as described herein.

### Formulations

Formulation 1: Acetaminophen oral suspension (32 mg/mL).

| Ingredient | Function | Per 5 mL | | Percent w/v |
|---|---|---|---|---|
| Acetaminophen | Active pharmaceutical | 160 | mg | 3.2 |
| micronized. | | | | |
| DV 90 - less than 50 | | | | |
| microns | | | | |
| Natural Agave Syrup | vehicle | 6200 | mg | 124.0 |
| Natural Acacia Gum | Dispersant | 50 | mg | 1.0 |
| Natural Citrus Extract | Adjust pH | 7.5 | mg | 0.15 |
| Organic Blue Berry | Flavoring agent | 15 | mg | 0.3 |
| Flavor | | | | |
| Purified Water | vehicle | 400 | mg | 8.0 |

The gum acacia was first dispersed in the water, which was heated to about 55° C. This dispersion was then cooled to room temperature. This formed a transparent, viscous liquid. To the gum acacia dispersion, the micronized acetaminophen was added under mixing to produce a uniform dispersion. This was added to the agave syrup under stirring. The natural citrus extract was added to adjust the pH, and the flavoring agent was added. This suspension met the 4-aminophenol impurity level (a degradant impurity of acetaminophen), the pH specification of 4.0 to 6.9, as well as physical, chemical, and microbiological standards as described in the USP.

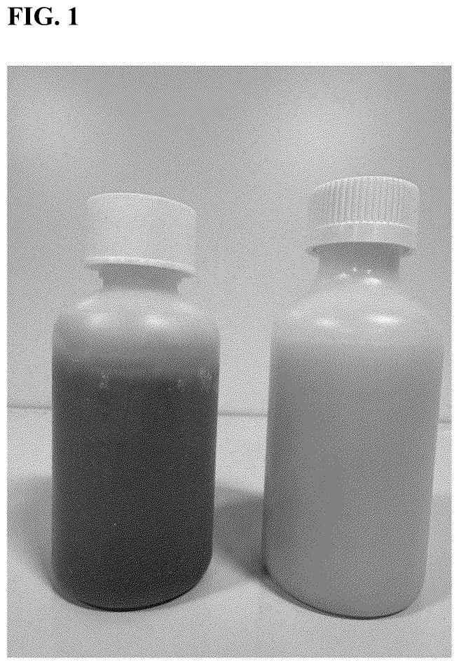

A comparison between a commercially available pharmaceutical formulation comprising acetaminophen, agave syrup, and water and Formulation 1 can be seen in FIG. 1. The commercially available formulation (left bottle) requires the vehicle to be heated in order to dissolve the acetaminophen, and subsequently this API separated out during storage as a creamed layer on top of the formulation, which is very difficult to redisperse and alters the color of the formulation. By comparison, Formulation 1 (right bottle) shows minimal to no separation upon storage, and is easily redispersible.

Formulation 2: Dextromethorphan hydrobromide and guaifenesin
oral suspension (1 mg/mL and 20 mg/mL, respectively).

| Ingredient | Function | Per 5 mL | | Percent w/v |
|---|---|---|---|---|
| Dextromethorphan | Active pharmaceutical | 5 | mg | 0.1 |
| Hydrobromide | | | | |
| Guaifenesin | Active pharmaceutical | 100 | mg | 2.0 |
| DV 90 - less than 50 | | | | |
| microns | | | | |
| Natural Agave Syrup | vehicle | 6250 | mg | 125.0 |
| Natural Acacia Gum | Dispersant | 50 | mg | 1.0 |
| Natural Citrus Extract | Adjust pH | 7.5 | mg | 0.15 |
| Organic Blue Berry | Flavoring agent | 15 | mg | 0.3 |
| Flavor | | | | |
| Purified Water | vehicle | 400 | mg | 8.0 |

The gum acacia was first dispersed in 6.5% w/v (e.g., 325 mg) of the water, which was heated to 55° C. This dispersion was then cooled to room temperature. This formed a transparent, viscous liquid. To the gum acacia dispersion, the micronized guaifenesin was added under mixing to produce a uniform dispersion. The remaining, second portion of the water (e.g., 1.5% w/v, or 75 mg) was heated to about 55° C. and used to dissolve the dextromethorphan hydrobromide to make a clear solution. Both the gum acacia dispersion comprising the guaifenesin and the dissolved dextromethorphan hydrobromide were added to the agave syrup under stirring. The natural citrus extract was added to adjust the pH, and the flavoring agent was added. The resulting formulation was a fine suspension that met physical, chemical, and microbiological standards.

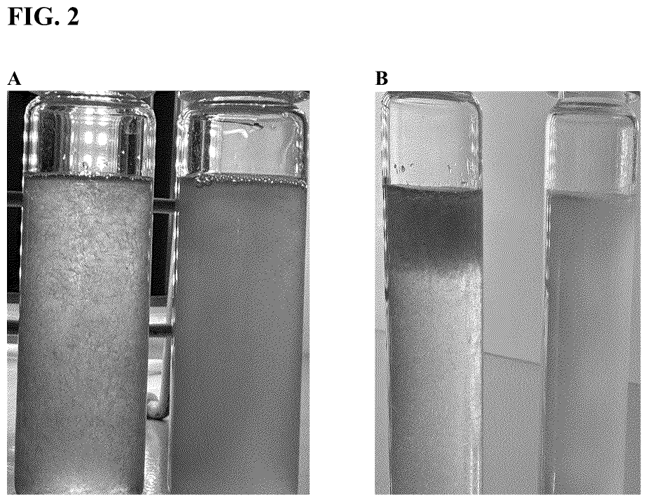

A comparison between a commercially available pharmaceutical formulation comprising guaifenesin, dextromethorphan hydrobromide, agave syrup, and water and Formulation 2 can be seen in FIG. 2. The commercially available formulation (left vial) requires the vehicle to be heated in order to dissolve the sparingly soluble guaifenesin, which also aids in dissolving the water soluble dextromethorphan hydrobromide which is suspended. Guaifenesin separated out during storage as crystalline needles, which remain after shaking. By comparison, Formulation 2 (right vial) shows minimal to no separation upon storage, and remains suspended without shaking. FIG. 2, panel A shows the formulations soon after mixing, while FIG. 2, panel B shows the formulations after 90 days of storage at room temperature, where the crystalline needles in the commercial formulation (left vial) have formed a solid layer on top of the formulation which is difficult to redisperse by shaking into a uniform suspension. By comparison, the vial of Formulation 2 (right vial) remains suspended within the formulation.

Formulation 3: Diphenhydramine hydrochloride
oral solution (2.5 mg/mL).

| Ingredient | Function | Per 5 mL | | Percent w/v |
|---|---|---|---|---|
| Diphenhydramine | Active pharmaceutical | 12.5 | mg | 0.25 |
| Hydrochloride | | | | |
| Natural Agave Syrup | vehicle | 6350 | mg | 127.0 |
| Natural Gum Acacia | Dispersant | 25 | mg | 0.5 |
| Natural Citrus Extract | Adjust pH | 7.5 | mg | 0.15 |
| Organic Blue Berry | Flavoring agent | 15 | mg | 0.3 |
| Flavor | | | | |
| Purified Water | vehicle | 400 | mg | 8.0 |

The gum acacia was first dispersed in 5% w/v (e.g., 250 mg) of the water, which was heated to about 55° C. This dispersion was then cooled to room temperature. This formed a transparent, viscous liquid. Note that the gum acacia in this formulation was used as a dispersant for insoluble ingredients that may have been present in the flavoring agent. The remaining, second portion of the water (e.g., 3% w/v, or 150 mg) was heated to about 55° C. and was used to dissolve the diphenhydramine hydrochloride to make a clear solution. Both the gum acacia dispersion and the dissolved diphenhydramine hydrochloride were added to the agave syrup under stirring. The natural citrus extract was added to adjust the pH, and the flavoring agent was added. The resulting formulation was a clear liquid that met physical, chemical, and microbiological standards.

Formulation 4: Dextromethorphan hydrobromide and chlorpheniramine
maleate oral solution (1.5 mg/mL and 0.2 mg/mL, respectively).

| Ingredient | Function | Per 5 mL | | Percent w/v |
|---|---|---|---|---|
| Dextromethorphan | Active pharmaceutical | 7.5 | mg | 0.15 |
| Hydrobromide | | | | |
| Chlorpheniramine | Active pharmaceutical | 1 | mg | 0.02 |
| Maleate | | | | |
| Natural Agave Syrup | vehicle | 6250 | mg | 125.0 |
| Natural Acacia Gum | Dispersant | 50 | mg | 1.0 |
| Natural Citrus Extract | Adjust pH | 7.5 | mg | 0.15 |
| Organic Blue Berry | Flavoring agent | 15 | mg | 0.3 |
| Flavor | | | | |
| Purified Water | vehicle | 400 | mg | 8.0 |

The gum acacia was first dispersed in 6% w/v (e.g., 300 mg) of the water, which was heated to about 55° C. This dispersion was then cooled to room temperature. This formed a transparent, viscous liquid. Note that the gum acacia in this formulation was used as a dispersant for insoluble ingredients that may have been present in the flavoring agent. A second portion of the water (e.g., 1% w/v, or 50 mg) was heated to 55° C. and was used to dissolve the dextromethorphan hydrobromide to make a clear solution. Similarly, a third portion of the water (e.g., 1% w/v, or 50 mg) was heated to 55° C. and was used to dissolve the chlorpheniramine maleate to make a clear solution. Both the gum acacia dispersion, the dissolved dextromethorphan hydrobromide, and the dissolved chlorpheniramine maleate were added to the agave syrup under stirring. The natural citrus extract was added to adjust the pH, and the flavoring agent was added. The resulting formulation was a clear liquid that met physical, chemical, and microbiological standards.

Formulation 5: Acetaminophen oral suspension (32
mg/mL) with agave syrup and tapioca syrup.

| Ingredient | Function | Per 5 mL | | Percent w/v |
|---|---|---|---|---|
| Acetaminophen | Active pharmaceutical | 160 | mg | 3.2 |
| micronized. | | | | |
| DV 90 - less than 50 | | | | |
| microns | | | | |
| Natural Agave Syrup | vehicle | 5580 | mg | 111.6 |
| Tapioca Syrup | vehicle | 620 | mg | 12.4 |
| Natural Acacia Gum | Dispersant | 50 | mg | 1.0 |
| Natural Citrus Extract | Adjust pH | 7.5 | mg | 0.15 |
| Organic Blue Berry | Flavoring agent | 15 | mg | 0.3 |
| Flavor | | | | |
| Purified Water | vehicle | 400 | mg | 8.0 |

The gum acacia was first dispersed in the water, which was heated to about 55° C. This dispersion was then cooled to room temperature. This formed a transparent, viscous liquid. To the gum acacia dispersion, the micronized acetaminophen was added under mixing to produce a uniform dispersion. This was added to the agave and tapioca syrup under stirring. The natural citrus extract was added to adjust the pH, and the flavoring agent was added. This suspension met the 4-aminophenol impurity level (a degradant impurity of acetaminophen), the pH specification of 4.0 to 6.9, as well as physical, chemical, and microbiological standards as described in the USP.

Formulation 6: Dextromethorphan hydrobromide and guaifenesin
oral suspension (1 mg/mL and 20 mg/mL, respectively)
with agave syrup and tapioca syrup.

| Ingredient | Function | Per 5 mL | | Percent w/v |
|---|---|---|---|---|
| Dextromethorphan | Active pharmaceutical | 5 | mg | 0.1 |
| Hydrobromide | | | | |
| Guaifenesin | Active pharmaceutical | 100 | mg | 2.0 |
| DV 90 - less than 50 | | | | |
| microns | | | | |
| Natural Agave Syrup | vehicle | 5625 | mg | 112.5 |
| Tapioca Syrup | vehicle | 625 | mg | 12.5 |
| Natural Acacia Gum | Dispersant | 50 | mg | 1.0 |
| Natural Citrus Extract | Adjust pH | 7.5 | mg | 0.15 |
| Organic Blue Berry | Flavoring agent | 15 | mg | 0.3 |
| Flavor | | | | |
| Purified Water | vehicle | 400 | mg | 8.0 |

The gum acacia was first dispersed in 6.5% w/v (e.g., 325 mg) of the water, which was heated to about 55° C. This dispersion was then cooled to room temperature. This formed a transparent, viscous liquid. To the gum acacia dispersion, the micronized guaifenesin was added under mixing to produce a uniform dispersion. The remaining, second portion of the water (e.g., 1.5% w/v, or 75 mg) was heated to about 55° C. and was used to dissolve the dextromethorphan hydrobromide to make a clear solution. Both the gum acacia dispersion comprising the guaifenesin and the dissolved dextromethorphan hydrobromide were added to the agave and tapioca syrup under stirring. The natural citrus extract was added to adjust the pH, and the flavoring agent was added. The resulting formulation was a fine suspension that met physical, chemical, and microbiological standards.

Formulation 7: Diphenhydramine hydrochloride oral solution
(2.5 mg/mL) with agave syrup and tapioca syrup.

| Ingredient | Function | Per 5 mL | | Percent w/v |
|---|---|---|---|---|
| Diphenhydramine | Active pharmaceutical | 12.5 | mg | 0.25 |
| Hydrochloride | | | | |
| Natural Agave Syrup | vehicle | 5715 | mg | 114.3 |
| Tapioca Syrup | vehicle | 635 | mg | 12.7 |
| Natural Gum Acacia | Dispersant | 25 | mg | 0.5 |
| Natural Citrus Extract | Adjust pH | 7.5 | mg | 0.15 |
| Organic Blue Berry | Flavoring agent | 15 | mg | 0.3 |
| Flavor | | | | |
| Purified Water | vehicle | 400 | mg | 8.0 |

The gum acacia was first dispersed in 5% w/v (e.g., 250 mg) of the water, which was heated to about 55° C. This dispersion was then cooled to room temperature. This formed a transparent, viscous liquid. Note that the gum acacia in this formulation was used as a dispersant for insoluble ingredients that may have been present in the flavoring agent. The remaining, second portion of the water (e.g., 3% w/v, or 150 mg) was heated to about 55° C. and was used to dissolve the diphenhydramine hydrochloride to make a clear solution. Both the gum acacia dispersion and the dissolved diphenhydramine hydrochloride were added to the agave and tapioca syrup under stirring. The natural citrus extract was added to adjust the pH, and the flavoring agent was added. The resulting formulation was a clear liquid that met physical, chemical, and microbiological standards.

Formulation 8: Pharmaceutical vehicle for the
preparation of a suspension comprising agave
syrup, water, and 1% w/v natural gum acacia.

| Ingredient | Function | Percent w/v |
|---|---|---|
| Natural Agave Syrup | vehicle | 127.0 |
| Natural Gum Acacia | dispersant | 1.0 |
| Purified Water | vehicle | 8.0 |

The gum acacia was first dispersed in the water, which was heated to about 55° C. This dispersion was then cooled to room temperature. This formed a transparent, viscous liquid. The gum acacia dispersion was then added into agave syrup under stirring to form a clear liquid. This met physical and microbiological standards. This vehicle can be used as a vehicle for preparing a suspension comprising poorly or sparingly soluble APIs. Without wishing to be bound by any particular theory, this type of vehicle may be useful for pharmaceutical compounding with a dispersion or suspension of ingredients.

The foregoing is illustrative of the present invention, and is not to be construed as limiting thereof. The invention is defined by the following claims, with equivalents of the claims to be included therein.

1. A pharmaceutical formulation for oral administration comprising:
a) one or more active pharmaceutical ingredient (API) present in a concentration from about 0.02 to about 5 weight/volume percentage concentration (% w/v) of the formulation;
b) a gum selected from gum acacia, gum tragacanth, gum almond, gum olibanum, gum ghatti, gum Albizia, and/or Moringa oleifera gum, present in a combined concentration from about 0.2 to about 2.0% w/v of the formulation;
c) a syrup selected from agave syrup, tapioca syrup, and/or maple syrup, present in a combined concentration from about 110 to about 130% w/v of the formulation; and
d) water present in a concentration from about 5 to about 45% w/v of the formulation.

2. The pharmaceutical formulation of claim 1, comprising:
a) one or more API present in a concentration from about 0.02 to about 5% w/v of the formulation;
b) gum acacia present in a concentration from about 0.2 to about 2.0% w/v of the formulation;
c) agave syrup present in a concentration from about 110 to about 130% w/v of the formulation; and
d) water present in a concentration from about 5 to about 45% w/v of the formulation.

3. The pharmaceutical formulation of claim 1, comprising:
a) one or more API present in a concentration from about 0.02 to about 5% w/v of the formulation;
b) gum acacia present in a concentration from about 0.2 to about 2.0% w/v of the formulation;
c) agave syrup and tapioca syrup present in a combined concentration from about 110 to about 130% w/v of the formulation; and
d) water present in a concentration from about 5 to about 45% w/v of the formulation.

4. The pharmaceutical formulation of claim 1, wherein the one or more API is water insoluble or sparingly soluble in water.

5. The pharmaceutical formulation of claim 4, wherein the one or more water insoluble or sparingly soluble in water API is suspended in the formulation.

6. The pharmaceutical formulation of claim 1, wherein each of the one or more API is a micronized API.

7. The pharmaceutical formulation of claim 1, wherein each of the one or more API has a DV90 from about 20 μm to about 100 μm.

8. The pharmaceutical formulation of claim 1.

9. The pharmaceutical formulation of claim 1, wherein the one or more API is an analgesic, a non-steroidal anti-inflammatory, an antihistamine, a cough suppressant, an expectorant, and/or a fever reducer, optionally wherein the one or more API is acetaminophen, guaifenesin, dextromethorphan hydrobromide, diphenhydramine hydrochloride, ibuprofen and/or chlorpheniramine maleate.

10. (canceled)

11. The pharmaceutical formulation of claim 1, wherein the formulation further comprises e) a flavoring agent, optionally wherein the flavoring agent is present a concentration from about 0.1 to about 1% w/v of the formulation, optionally wherein the flavoring agent is blueberry flavor.

12.-13. (canceled)

14. The pharmaceutical formulation of claim 1, wherein the formulation further comprises f) a pH buffer, optionally wherein the pH buffer is present a concentration from about 0.1 to about 1% w/v of the formulation, optionally wherein the pH buffer is citrus extract, optionally wherein the pH of the formulation is from about 2 to about 7, from about 4.0 to about 6.9, or from about 2.3 to about 6.5.

15.-19. (canceled)

20. The pharmaceutical formulation of claim 1, wherein the viscosity of the formulation is less than about 1400 centipoise at about 22° C. optionally less than about 900 centipoise at about 22° C., optionally less than about 450 centipoise at about 22° C.

21.-22. (canceled)

23. The pharmaceutical formulation of claim 14, wherein the gum, the syrup, the flavoring agent, and/or the pH buffer are natural ingredients.

24. The pharmaceutical formulation of claim 14, comprising:
a) micronized acetaminophen present in a concentration of 3.2% w/v;
b) agave syrup present in a concentration of 124% w/v;
c) gum acacia present in a concentration of 1.0% w/v;
d) water present in a concentration of 8% w/v;
e) blueberry flavor present in a concentration of 0.3% w/v; and
f) citrus extract present in a concentration of 0.15% w/v;
or
a) dextromethorphan hydrobromide present in a concentration of 0.1% w/v and micronized guaifenesin present in a concentration of 2.0% w/v;
b) agave syrup present in a concentration of 125% w/v;
c) gum acacia present in a concentration of 1.0% w/v;
d) water present in a concentration of 8.0% w/v;
e) blueberry flavor present in a concentration of 0.3% w/v; and
f) citrus extract present in a concentration of 0.15% w/v;
or
a) diphenhydramine hydrochloride present in a concentration of 0.25% w/v;
b) agave syrup present in a concentration of 127% w/v;
c) gum acacia present in a concentration of 0.5% w/v;
d) water present in a concentration of 8.0% w/v;
e) blueberry flavor present in a concentration of 0.3% w/v; and
f) citrus extract present in a concentration of 0.15% w/v;
or
a) dextromethorphan hydrobromide present in a concentration of 0.15% w/v and chlorpheniramine maleate present in a concentration of 0.02% w/v;
b) agave syrup present in a concentration of 125% w/v;
c) gum acacia present in a concentration of 1.0% w/v;
d) water present in a concentration of 8.0% w/v;
e) blueberry flavor present in a concentration of 0.3% w/v; and
f) citrus extract present in a concentration of 0.15% w/v;
or
a) micronized acetaminophen present in a concentration of 3.2% w/v;
b) agave syrup present in a concentration of 111.6% w/v and tapioca syrup is present in a concentration of 12.4% w/v;
c) gum acacia present in a concentration of 1.0% w/v;
d) water present in a concentration of 8.0% w/v;
e) blueberry flavor present in a concentration of 0.3% w/v; and
f) citrus extract present in a concentration of 0.15% w/v;
or
a) dextromethorphan hydrobromide present in a concentration of 0.1% w/v and guaifenesin present in a concentration of 2.0% w/v;
b) agave syrup present in a concentration of 112.5% w/v and tapioca syrup is present in a concentration of 12.5% w/v;
c) gum acacia present in a concentration of 1.0% w/v;
d) water present in a concentration of 8.0% w/v;
e) blueberry flavor present in a concentration of 0.3% w/v; and
f) citrus extract present in a concentration of 0.15% w/v;
or
a) diphenhydramine hydrochloride present in a concentration of 0.25% w/v;
b) agave syrup present in a concentration of 114.3% w/v and tapioca syrup is present in a concentration of 12.7% w/v;
c) gum acacia present in a concentration of 0.5% w/v;
d) water present in a concentration of 8.0% w/v;
e) blueberry flavor present in a concentration of 0.3% w/v; and
f) citrus extract present in a concentration of 0.15% w/v.

25.-30. (canceled)

31. A pharmaceutical vehicle for oral administration, comprising:
a) a gum selected from gum acacia, gum tragacanth, gum almond, gum olibanum, gum ghatti, gum Albizia, and/or Moringa oleifera gum present in a combined concentration from about 0.2 to about 1.5% w/v of the formulation;
b) a syrup selected from agave syrup, tapioca syrup, and/or maple syrup present in a combined concentration from about 110 to about 130% w/v of the formulation;
c) water present in a concentration from about 5 to about 45% w/v of the formulation; and
wherein the pharmaceutical vehicle is suitable for suspending one or more water soluble, sparingly soluble, and/or water insoluble API.

32. The pharmaceutical vehicle of claim 31, comprising:
a) gum acacia present in a concentration of 1.0% w/v;
b) agave syrup present in a concentration of 127% w/v; and
c) water present in a concentration of 8.0% w/v.

33. A method of making the pharmaceutical vehicle of claim 31, the method comprising:
i) dissolving the gum in the water to form a viscous liquid, wherein the water is heated; and
ii) stirring the viscous liquid into the syrup to form a pharmaceutical formulation for oral administration;
optionally, wherein the water is heated to about 40° C. to about 60° C.;
optionally wherein the gum is gum acacia and the syrup is agave syrup and/or tapioca syrup.

34.-35. (canceled)

36. A method for manufacturing a pharmaceutical formulation for oral administration, the method comprising:
i) dissolving a gum selected from gum acacia, gum tragacanth, gum almond, gum olibanum, gum ghatti, gum Albizia, and/or Moringa oleifera gum in heated water to form a viscous liquid;
ii) stirring one or more micronized API into the viscous liquid to form a uniform dispersion; and
iii) stirring the uniform dispersion into a syrup selected from agave syrup, tapioca syrup, and/or maple syrup to form a pharmaceutical formulation for oral administration;
or
i) dissolving a gum selected from gum acacia, gum tragacanth, gum almond, gum olibanum, gum ghatti, gum Albizia, and/or Moringa oleifera gum in a first portion of heated water to form a viscous liquid;
ii) stirring a first micronized API into the viscous liquid to form a uniform dispersion;
iii) dissolving a second API in a second portion of heated water to form a solution; and
iv) stirring the uniform dispersion and the solution into a syrup selected from agave syrup, tapioca syrup, and/or maple syrup to form a pharmaceutical formulation for oral administration;
or
i) dissolving a gum selected from gum acacia, gum tragacanth, gum almond, gum olibanum, gum ghatti, gum Albizia, and/or Moringa oleifera gum in a first portion of heated water to form a viscous liquid;
ii) dissolving one or more API in a second portion of heated water to form a solution; and
iii) stirring the viscous liquid and the solution into a syrup selected from agave syrup, tapioca syrup, and/or maple syrup to form a pharmaceutical formulation for oral administration;
or
i) dissolving a gum selected from gum acacia, gum tragacanth, gum almond, gum olibanum, gum ghatti, gum Albizia, and/or Moringa oleifera gum in a first portion of heated water to form a viscous liquid;
ii) dissolving a first API in a second portion of heated water to form a first solution;
iii) dissolving a second API in a third portion of heated water to form a second solution; and
iv) stirring the uniform dispersion, the first solution, and the second solution into a syrup selected from agave syrup, tapioca syrup, and/or maple syrup to form a pharmaceutical formulation for oral administration.

37.-39. (canceled)

40. The method of claim 33, wherein forming the pharmaceutical formulation further comprises stirring a pH buffer and/or a flavoring agent into the syrup.

41. The method of claim 33, wherein the heated water, the first portion of the heated water, the second portion of the heated water, and the third portion of the heated water are about 40° C. to about 60° C.

42. A method of delivering an API to a subject in need thereof, the method comprising orally administering to the subject the pharmaceutical formulation of claim 1, thereby delivering the API to the subject.

43.-45. (canceled)
